(19) United States
(12) Patent Application Publication
(10) Pub. No.: US 2005/0055045 A1
(43) Pub. Date: Mar. 10, 2005

(54) Composite medical devices

(75) Inventors: Robert B. DeVries, Northborough, MA (US); Kristian DiMatteo, Waltham, MA (US); Steven Walak, Natick, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/659,490

(22) Filed: Sep. 10, 2003

(51) Int. Cl. A61M 29/00

(52) U.S. Cl. 606/200

(57) ABSTRACT

Composite medical devices for placement within a body vessel are disclosed. A composite medical device may include an intravascular filter, stent, stent graft, or other implantable device having a relatively stiff outer member, and an elastic inner member. In some embodiments, the composite medical device may comprise an intravascular filter having a plurality of elongated legs formed from a composite material. In other embodiments, the composite medical device may comprise a composite stent or stent graft formed of composite threads. Selective portions of the outer member can be removed to expose the elastic inner member, thereby forming a flexibility region on the device.

### FIELD OF THE INVENTION

The present invention relates generally to devices implantable within a body lumen. More specifically, the present invention pertains to composite medical devices implantable within a body vessel.

### BACKGROUND OF THE INVENTION

Implantable medical devices such as vena cava filters, stents, and stent grafts are used in treating vascular disease at various locations in the body. Such devices are typically inserted percutaneously into the body via an introducer catheter, and advanced to a desired location within a body vessel. Once positioned, the device is then removed from within the introducer catheter, causing the device to self-expand within the vessel.

Access to the blood vessels is generally accomplished through a puncture opening formed in the femoral, jugular, or antecubital veins. Since relatively large introducer catheters are required to transport the filter or stent, access to the blood vessel is generally attained through the femoral or jugular veins. In some cases, access to smaller veins such as an antecubital vein is impossible since the profile of the introducer catheter and enclosed medical device prevents insertion.

### SUMMARY OF THE INVENTION

The present invention relates generally to composite medical devices implantable within a body vessel. In one exemplary embodiment of the present invention, a composite medical device may comprise an intravascular filter having an apical head, and a plurality of elongated legs formed of an outer member comprising a first material and an inner member comprising a second material different from the first material. In certain embodiments, the first material may comprise a relatively stiff material, whereas the second material may comprise a relatively elastic material.

The elongated legs may be configured to bend or flex in an outswept manner when placed within a body lumen such as a blood vessel. In some embodiments, the inner member may comprise a shape-memory material such as a nickel-titanium alloy (Nitinol) configured to bend from a substantially straight position to an outswept position when exposed to a particular temperature. One or more zigzag regions may be formed by removing a portion of the outer member from each elongated filter leg. The zigzag regions may be staggered at various locations along the length of each elongated filter leg to reduce the profile of the filter when collapsed within an introducer catheter.

In certain embodiments, the intravascular filter may include a hook region on each of the elongated filter legs configured to engage the wall of the vessel. The hook region may include a main section, a reversibly bent section, and a pointed tip section. The hook region may be formed by removing a proximal portion of the elongated leg to expose the inner member, which in some embodiments may comprise a superelastic material having shape-memory properties. As with the zigzag regions, each hook region may be configured to bend or flex to a predefined shaped when exposed to a particular temperature.

In another exemplary embodiment of the present invention, a composite medical device may comprise a stent or stent graft having a number of individual threads formed from composite wire members. As with the filter embodiments, each thread may be formed from an outer member comprising a relatively stiff material, and an inner member comprising a relatively elastic material such as superelastic and/or shape-memory nickel-titanium alloy.

The composite stent may include a number of flexibility regions formed by removing a portion of the stiff outer member to expose the elastic inner member. In certain embodiments, one or more flexibility regions may be formed on the ends of the stent. In use, the flexibility regions act as a hinge, allowing the device to be radially collapsed into relatively small delivery devices while maintaining the desired expansion characteristics of the device.

### BRIEF DESCRIPTION OF THE DRAWINGS

FIG. 1 is a perspective view of a composite intravascular filter in accordance with an exemplary embodiment of the present invention;

FIG. 2 is an exploded view of the apical head illustrated in FIG. 1, showing the insertion of the distal end of each elongated leg within the apical head;

FIG. 3 is a cross-sectional view of the intravascular filter of FIG. 1 along line 3-3;

FIG. 4 is a longitudinal cross-sectional view of a portion of the elongated leg of FIG. 3 along line 4-4;

FIG. 5 is a perspective view of a composite intravascular filter in accordance with another exemplary embodiment of the present invention, wherein the zigzag regions along each elongated leg are longitudinally offset from each other;

FIG. 6 is a cross-sectional view of the intravascular filter of FIG. 5 along line 6-6;

FIG. 7 is a longitudinal cross-sectional view of a portion of the elongated leg of FIG. 6 along line 7-7;

FIG. 8 is a perspective view of an intravascular filter in accordance with another exemplary embodiment of the present invention, wherein a portion of the outer member is removed to form a hook region on each elongated leg;

FIG. 9 is a view of the proximal portion of one of the elongated legs illustrated in FIG. 8, showing the hook region engaged within the wall of a body vessel;

FIG. 10 is a perspective view of a composite intravascular filter in accordance with another exemplary embodiment of the present invention, wherein a portion of the outer member is removed to form one or more zigzag regions;

FIG. 11 is a partial cross-sectional view of a raw composite member used to form an elongated filter leg in accordance with an exemplary embodiment of the present invention;

FIG. 12 is a partial cross-sectional view of the composite member of FIG. 11, showing the composite member bent into a zigzag shape;

FIG. 13 is a partial cross-sectional view of the bent composite member of FIG. 12, showing the outer member removed to expose the inner member at the zigzag region;

FIG. 14 is a partial cross-sectional view of the composite member of FIG. 13, showing the zigzag region compressed into a straight position;

FIG. 15 is a perspective view of a composite stent in accordance with an exemplary embodiment of the present invention;

FIG. 16 is a cross-sectional view of one of the threads illustrated in FIG. 15; and

FIG. 17 is an exploded view of one of the end threads illustrated in FIG. 15.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, materials and manufacturing processes are illustrated for the various elements, those skilled in the art will recognize that many of the examples provided have suitable alternatives that may be utilized.

Although discussed with specific reference to intravascular filters and stents in the particular embodiments described herein, the invention may be applicable to a variety of other medical devices implantable within a body vessel of a patient. For example, certain aspects of the present invention may be applicable to guidewires, catheters (e.g. balloon, stent delivery, etc.), drive shafts for rotational devices such as atherectomy catheters and IVUS catheters, endoscopic devices, laparoscopic devices, retrieval baskets, embolic protection devices, snares, and other such devices.

In at least some embodiments, the invention is directed to composite medical devices, and/or wire members for use therein, that can include a relatively elastic inner member and a relatively stiff outer member. In certain embodiments, both the inner and outer members may be formed of a metal or metal alloy, as described herein. In some embodiments, the composite medical device may be constructed from an outer member including a first metallic material, and an inner member including a second metallic material different, and in some cases more flexible, than the first material. A portion of the metallic outer member may be removed from the composite member to expose a portion of the metallic inner member. Removal of the outer member to expose portions of the inner member can be used to impart flexibility to the composite medical device, as desired. For example, portions of the composite medical device can be rendered more flexible by removing all or a portion of the outer member. Conversely, portions of the medical device can be rendered stiff by maintaining the outer member.

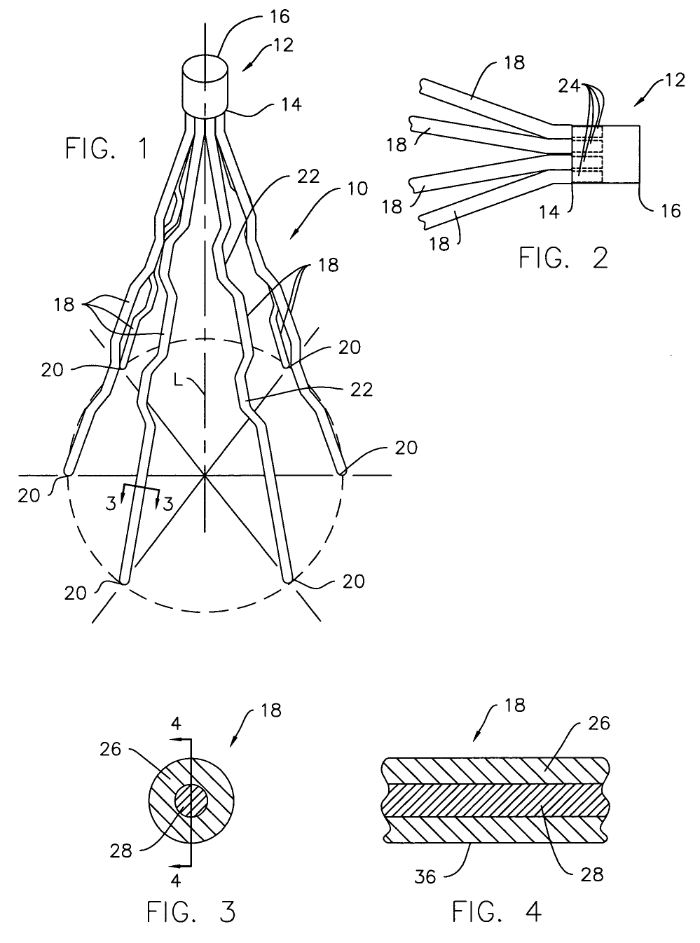

Referring now to FIG. 1, a composite medical device 10 in accordance with an exemplary embodiment of the present invention will now be described. Composite medical device 10, illustratively an intravascular filter, comprises an apical head 12 having a proximal portion 14 and a distal portion 16, and a plurality of elongated legs 18 each having a proximal end 20 and a distal end (not shown). As is discussed in greater detail below, the elongated legs 18 are configured to bend or flex in an outswept manner when deployed within a body lumen such as the inferior vena cava.

In a deployed position illustrated in FIG. 1, each of the elongated legs 18 may be symmetrically spaced about a central longitudinal axis L in a generally conical-shaped configuration. The elongated legs 18 are collectively arranged about the longitudinal axis L such that the distal end of each elongated leg 18 converges at the apical head 12 to form an apex, and the proximal end 20 of each elongated leg 18 forms a mouth for filtering embolic debris contained within the vessel.

One or more zigzag regions 22 disposed along the length of each leg 18 are configured to impart flexibility to the elongated legs 18, and to increase the surface area of the intravascular filter 10. In the exemplary embodiment illustrated in FIG. 1, each zigzag region 22 is bent along a plane tangential to the conical configuration of the elongated legs 18 such that when the intravascular filter 10 is collapsed within a delivery device (e.g. an introducer catheter), the profile of the intravascular filter 10 at each zigzag region 22 is substantially the same as the profile along the remaining portion of the elongated legs 18.

FIG. 2 is an exploded view of the apical head illustrated in FIG. 1, showing the insertion of the distal end 24 of each elongated leg 18 within the proximal portion 14 of the apical head 12. As shown in FIG. 2, the distal end 24 of each elongated leg 18 may be configured in size and shape to fit within a corresponding hole or bore formed in the proximal portion 14 of the apical head 12. The distal end 24 of each elongated leg 18 can be secured within each hole or bore by any suitable attachment means, including soldering, welding, crimping, and/or an adhesive. Those of skill in the art will realize, however, that other methods could be used to secure the distal end 24 of each elongated leg 18 to the apical head 12, as desired.

Although the apical head 12 illustrated in FIG. 1 is substantially cylindrical in shape, it is to be understood that various geometric shapes may be utilized. For example, the apical head 12 may have a conical or pyramid shape. Moreover, in some embodiments, the apical head 12 may include an inner lumen (not shown) configured to slidably receive a guidewire, guide catheter, or other elongated member.

In certain embodiments, the distal end 24 of each elongated leg 18 may be reduced in diameter or tapered to decrease the profile of the intravascular filter 10 at the apical head 12. As shown in FIG. 2, for example, the outer portion of each elongated leg 18 at distal end 24 may be removed, allowing the use of smaller holes or bores in the apical head 12. Removal of the outer portion of each elongated leg 18 at distal end 24 may be accomplished by grinding, turning, stripping or etching the outer portion of each elongated leg 18.

FIG. 3 is a cross-sectional view of the intravascular filter 10 along line 3-3 in FIG. 1, showing the composite structure of one of the elongated legs 18. As shown in FIG. 3, each of the elongated legs 18 may comprise an outer member 26 formed of a first material, and an inner core member 28 formed of a second material different from the first material. The outer member 26 may comprise a relatively stiff material, whereas the inner core member 28 may comprise a relatively elastic material. In certain embodiments, for example, the inner core member 28 may be formed of a material having superelastic or pseudo-elastic characteristics such as nickel-titanium alloy (Nitinol), beta titanium alloy, titanium-palladium alloy (Ti—Pd), titanium-platinum alloy (Ti—Pt), or nickel-titanium-copper alloy (Ni—Ti—Cu), whereas the outer member 26 may be formed of a relatively stiff material such as stainless steel, gold, molybdenum, platinum, titanium, tungsten, Elgiloy, L605, MP35N, Ta-10W, 17-4PH, Aeromet 100, cobalt-chrome alloy or cobalt alloys, metal glass alloys, and refractory metal alloys.

FIG. 4 is a longitudinal cross-sectional view of a portion of the elongated leg 18 along line 4-4 shown in FIG. 3. As shown in FIG. 4, the elongated leg 18 may assume a substantially straight configuration at room temperature, allowing the intravascular filter 10 to be easily loaded into the delivery device.

In some embodiments of the present invention, the outer surface 36 of each elongated leg 18 may include a radiopaque material configured to permit monitoring of the intravascular filter 10 with a fluoroscopic monitor located outside of the patient's body. Radiopaque, materials are understood to be materials capable of producing a relatively bright image on a fluoroscopic screen or other imaging device during a medical procedure. This relatively bright image aids the user of a device incorporating the radiopaque material in determining the location and deployment status of the device. Examples of suitable radiopaque materials include gold, palladium, platinum, tungsten, or polymers loaded with a radiopaque filler.

The outer surface 36 of each elongated leg 18 may also be coated with a suitable biocompatible polymeric material to facilitate transport and deployment of the intravascular filter 10 within the body. Examples of biocompatible polymeric materials include polyacrylic acid, polytetraflouroethylene (PTFE), paralyene, polycaprolactone, polycarboxylic acid, polyamide, polyvinyl ether, polyurethane and polyorthoesters. Polyacrylic acid is commercially available from Boston Scientific Corporation of Natick, Mass. under the trade name HYDROPASS. Furthermore, each elongated leg 18 may also include an anti-thrombogenic material to reduce insertion site thrombosis within the body vessel.

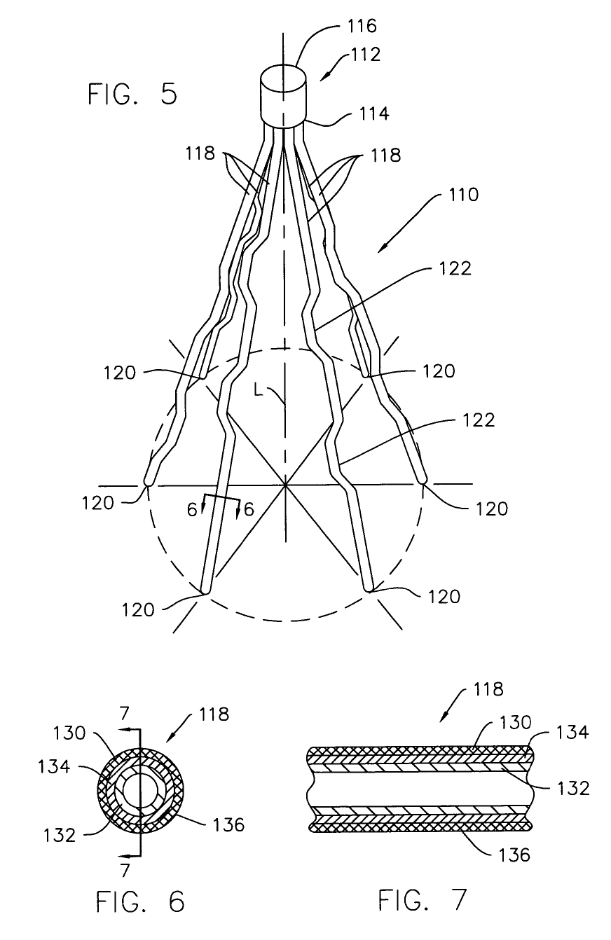

FIG. 5 is a perspective view of another exemplary embodiment of an intravascular filter 110 in accordance with the present invention, wherein one or more zigzag regions 122 along each elongated leg 118 are longitudinally offset from each other. Intravascular filter 110 comprises an apical head 112 having a proximal portion 114 and a distal portion 116, and a plurality of elongated legs 118 configured to bend or flex when deployed within a body vessel.

Each elongated leg 118 may include one or more zigzag regions 122 disposed along a plane tangential to the conical configuration of the elongated legs 118. The one or more zigzag regions 122 may be staggered at various locations along the length of each elongated leg 118 such that, when the intravascular filter 110 is in a collapsed position within the delivery device, the one or more zigzag regions 122 on a particular elongated leg 118 do not interfere with the one or more zigzag regions 122 on an adjacent (i.e. neighboring) elongated leg 118. This staggered configuration may, under certain circumstances, reduce the profile of the device when collapsed within the delivery device, and prevents leg-crossing when expanded within the body vessel.

As shown in FIGS. 6-7, each of the elongated legs 118 may be formed of a composite material comprising an outer tubular member 130 formed of a first material, an inner tubular member 132 formed of a second material different from the first material, and a middle member 134 made of a third material different from the first and second materials. In some embodiments, for example, the outer, inner and middle members 130, 132, 134 may be formed of materials have different properties such as stiffness, hardness, lubricity, and/or radiopacity that can be selected for a particular application.

In certain embodiments, one or more of the tubular members 130, 132, 134 may include a shape-memory and/or superelastic alloy such as nickel-titanium. In one exemplary embodiment, the outer member 130 may be formed of a superelastic, shape-memory material, whereas the middle and inner tubular members 134, 132 may be formed of relatively stiff materials such as stainless steel, gold, molybdenum, platinum, titanium, tungsten, Elgiloy, L605, MP35N, Ta-10W, 17-4PH, Aeromet 100, cobalt-chrome alloy, metal glass alloy, and refractory metal alloy. The shape-memory outer member 130 can be heat-set with an outwardly bent shape configured to expand the elongated legs 118 in an outward direction when deployed in the body vessel. In use, the exposure of the outer surface 136 of the outer tubular member 130 to temperature within the body vessel causes the shape-memory material to revert to its predefined (i.e. bent) shape. As with any of the other embodiments described herein, the outer surface 136 of the outer tubular member 130 may also include a radiopaque and/or polymeric coating.

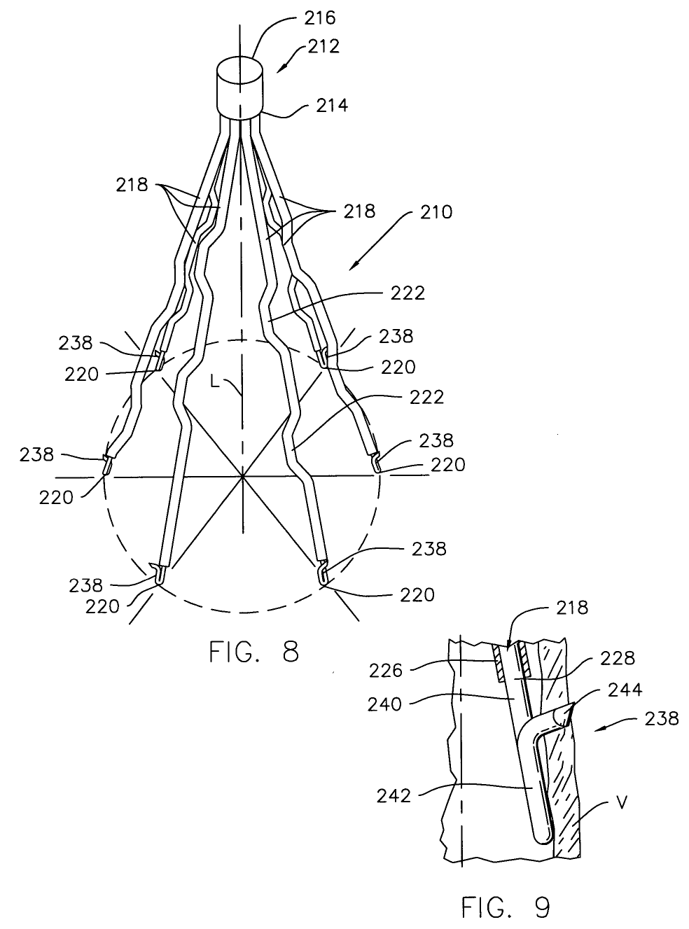

FIG. 8 is a perspective view of another exemplary embodiment of an intravascular filter 210 in accordance with the present invention, wherein a portion of the proximal end 220 of each elongated filter leg 218 is removed to form a flexible hook region 238. Intravascular filter 210 may comprise an apical head 212 having a proximal end 214 and a distal end 216, and a plurality of elongated filter legs 218 configured to bend or flex when deployed within a body vessel. In the exemplary embodiment illustrated in FIG. 8, the elongated legs 218 are formed of solid members, similar to that depicted in FIGS. 1-4. It should be understood, however, that any of the various leg embodiments described herein can be utilized.

Intravascular filter 210 may further include a hook region 238 at or near the proximal end 220 of each of elongated leg 218 that can be configured to pierce the vessel wall, securing the intravascular filter 210 within the vessel. As shown in FIG. 9, each hook region 238 may be formed by removing a proximal portion of the outer member 226 to expose the inner member 228. The hook region 238 may be formed of a two-way shape-memory alloy, such as a binary nickel-titanium alloy, configured to bend or flex to a predefined shape when exposed to a particular temperature within the body vessel, but remain straight when restrained by a sheath.

In the embodiment illustrated in FIGS. 8-9, the hook region 238 may be configured to revert to a predefined shape having a main section 240, and a reversibly bent section 242 bent through an angle of about 180° in the plane tangential to the conical configuration of the elongated leg 218 and disposed approximately parallel and contiguous to the main section 240. Hook region 238 may further include a pointed tip section 244 or other piercing means configured to engage the vessel wall. The contiguous main and reversibly bent sections 240, 242 of the hook region 238 form a pad or landing for limiting or restricting the penetration depth of the pointed tip section 244 into vessel wall.

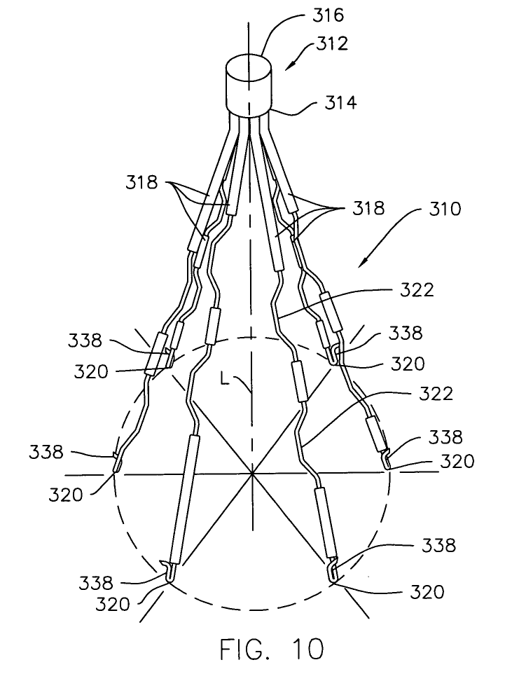

FIG. 10 is a perspective view of another exemplary embodiment of an intravascular filter 310 in accordance with the present invention, wherein one or more longitudinally offset zigzag regions 322 are formed by removing a portion of each elongated filter leg 318. Intravascular filter 310 may be configured similar to intravascular filter 210, comprising an apical head 312 having a proximal end 314 and a distal end 316, and a plurality of elongated filter legs 318 formed of a composite material configured to bend or flex when deployed within a body vessel. A hook region 338 on the proximal end 320 of each elongated leg 318 may be configured to pierce the body vessel and secure the intravascular filter 310 within the body vessel.

The one or more longitudinally offset zigzag regions 322 may be formed by removing the outer member at various locations along the length of the elongated member 318 to expose the inner member. The inner member may be formed of a shape-memory material configured to deform to a predefined shape when subjected to a particular temperature within the body vessel. For example, the exposed portions of the inner member may be configured to bend or flex from a substantially straight position when disposed within the delivery device to a zigzag position when deployed within the body vessel. The outer member can also be removed along the entire length of the elongated filter leg 318 but retain proximal apical head 312, if desired.

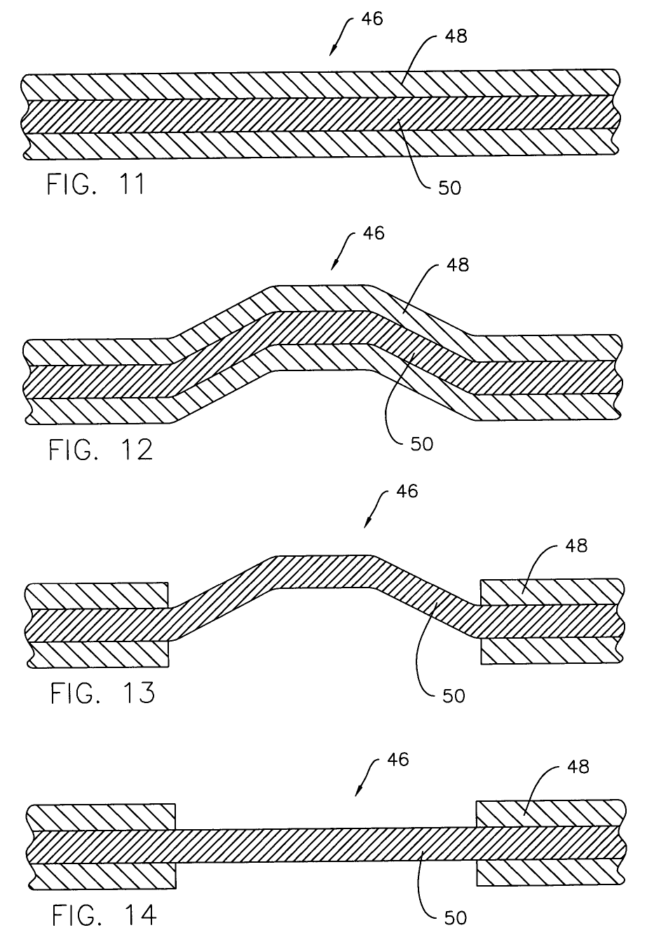

Referring now to FIG. 11, an exemplary method of removing the outer member to form the one or more zigzag regions may include the process of taking a raw composite member 46 comprising an outer member 48 formed of a relatively stiff material, and an inner member 50 formed of a relatively elastic material (e.g. superelastic Nitinol), and subjecting the composite member 46 to an external force to impart a shape thereto. As shown in FIG. 12, for example, the composite member 46 can be bent to form a zigzag shape. Nitinol elements should then be subjected to a thermal shape setting heat treatment. Heat treatment in the range of 350C to 600C for 2 to 50 minutes, preferably between 425C and 550C for 5 to 30 minutes, will establish the “remembered” shape for the nitinol element.

Once the desired shape has been formed, the composite member 46 is then subjected to an etching step wherein a portion of the outer member 48 can be mechanically, chemically and/or electrically removed to expose a portion of the inner member 50, as shown in FIG. 13. To remove material from the zigzag shape(s), desired sections of the composite member 46 may be submersed in a dip bath containing an acidic solution capable of chemically etching the outer member 48 to expose the inner member 50. Current can be applied through the dip bath to etch the outer member 48. The rate at which this process occurs can be varied depending on the amount of current sent through the dip bath into the inner member 50.

In an alternative method, portions of the outer member 48 can be mechanically removed to expose the inner member 50 and form the zigzag region(s). In certain embodiments, for example, a jacket stripper can be utilized to strip away the outer member 48 to expose the inner member 50. In other embodiments, the outer member 48 can be removed from the desired area by a centerless grinding technique.

Once formed, the composite member 46 can then be attached to other similarly produced members to form the intravascular filter. As shown in FIG. 14, when the composite member 46 is compressed within a delivery device (not shown) such as an introducer catheter, the inner member 50 compresses to a substantially straight position, allowing the intravascular filter to be loaded into a delivery device having a smaller profile. Upon deployment within the body, the exposed portion(s) of the inner member 50 revert to their predefined shape.

Although the use of six elongated legs is specifically illustrated in the aforesaid embodiments, other configurations have been envisioned. For example, an intravascular filter in accordance with the present invention may include three, four, five, seven, eight, etc. elongated legs that can be expanded within a vessel to collect and store embolic debris contained in the blood. Moreover, while the various elongated legs described herein are shown having a circular cross-section, other shapes have been contemplated. In some embodiments, for example, the elongated legs may be formed of ribbon members having a rectangular cross-section.

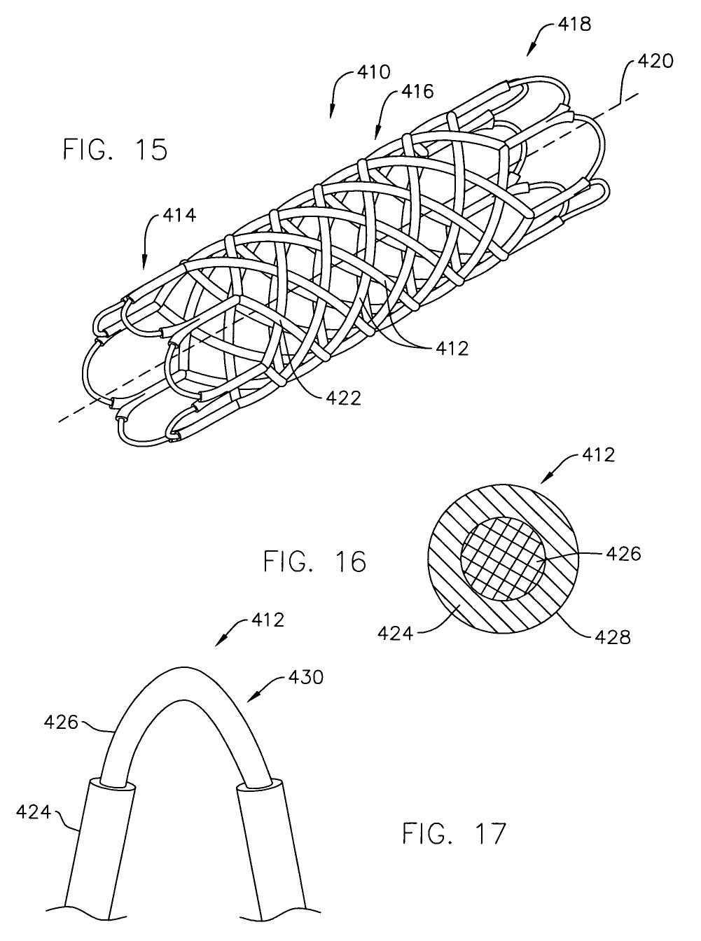

FIG. 15 is a perspective view of another composite medical device 410 in accordance with another exemplary embodiment of the present invention. Composite medical device 410, illustratively a stent or stent graft, includes a number of individual wire threads 412 configured to radially expand the stent 410 when deployed within a body vessel. In an expanded position illustrated in FIG. 15, stent 410 assumes a substantially tubular shape, having a first end portion 414, a middle portion 416, and a second end portion 418.

The individual threads 412 forming the middle portion 416 of the stent 410 may be arranged generally in two sets of parallel helices wound in opposite directions about a common longitudinal axis 420 of the stent 410. The individual threads 412 may intersect each other in an overlapping pattern at a number of interstices 422. The interstices are configured to permit individual threads 412 to move with respect to each other, allowing the stent 410 to radially expand and axially shorten when deployed in the body. The threads 412 forming the end portions 414,418 of the stent 410 may be oriented in a direction substantially parallel to the longitudinal axis 420 of the stent 410, and may have a closed end configuration.

In one aspect of the present invention, the threads 412 may be constructed from a composite structure employing multiple materials having certain flexibility and/or radiopacity characteristics. As shown in FIG. 16, for example, at least some of the threads 412 may comprise an outer member 424 formed of a first material, and an inner member 426 formed of a second material different from the first material. The outer member 424 may comprise a relatively stiff material, whereas the inner member 426 may comprise a relatively elastic material. In certain embodiments, for example, the inner member 426 may be formed of a material having superelastic or pseudo-elastic characteristics such as nickel-titanium alloy (Nitinol), beta titanium alloy, titanium-palladium alloy (Ti—Pd), titanium-platinum alloy (Ti—Pt), or nickel-titanium-copper alloy (Ni—Ti—Cu), whereas the outer member 424 may be formed of a relatively stiff material such as stainless steel, gold, molybdenum, platinum, titanium, tungsten, Elgiloy, L605, MP35N, Ta-10W, 17-4PH, Aeromet 100, cobalt-chrome alloy or cobalt alloys, metal glass alloys, and refractory metal alloys. As with other embodiments described herein, the outer surface 428 of the outer member 424 may also include a radiopaque and/or polymeric coating, if desired. Additionally, in at least some embodiments, all or a portion of the outer and/or inner members 424,426 may be doped with, made of, coated or plated with, or otherwise include a radiopaque material.

While the threads depicted in FIGS. 15-16 have a substantially solid cross-sectional area, other configurations are possible. In one alternative embodiment, for example, tubular composite wire members may be used to form the various threads of the stent, similar to that discussed above with respect to filter legs illustrated in FIGS. 5-7. Moreover, while the various threads described herein are shown having a substantially circular cross-section, other shapes have been contemplated. In some embodiments, for example, the threads may be formed of ribbon members having a rectangular cross-section.

FIG. 17 is an exploded view of one of the threads 412 forming the first end portion 414 of stent 410. As can be seen in FIG. 17, a flexibility region 430 may be formed each thread 412 by removing all or a portion of the outer member 424, exposing the inner member 426 at the location where the threads 412 are subjected to a high degree of deformation during loading and delivery. In use, this flexibility region 430 acts as a hinge, allowing the stent 410 to be compressed into smaller delivery devices without significantly affecting the radial force that the stent 410 exerts on the wall of the vessel. In some cases, the relatively stiff outer member 428 may be used to impart a greater amount of radial force to the threads than a similarly dimensioned stent employing a single material.

The wire members used to form the various stent threads may be fabricated from any number of suitable processes. In one exemplary process, the threads can be formed by forming a bore through the center portion of a rod comprised of the outer member material, and then inserting a smaller, mating rod of inner core member material through the bore. The two members may then be swaged and drawn, forming a metallurgical bond between the different materials. The formed composite member can the be subjected to various heat treating steps, if desired, to anneal, harden, and/or impart superelastic or shape-memory properties to the material.

Once the desired shape has been imparted, the composite member may be fabricated into the desired device configuration. In one embodiment, for example, the formed composite member may be braided about a mandrel to form the general structure illustrated in FIG. 15, and then subjected to heat treatment to impart the desired mechanical properties to the stent. Selective regions of the device may then be selectively treated to remove the stiff outer cladding, thereby exposing the elastic inner member. In certain embodiments, for example, a chemical, electro-chemical, or grinding process may be employed to selective remove portions of the outer member, as desired.

Having thus described the several embodiments of the present invention, those skilled in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

1. A composite medical device, comprising:
a composite elongated member formed from an outer member comprising a first material and an inner member comprising a second material different from the first material, wherein the second material is more elastic than the first material; and
at least one flexibility region formed on said composite elongated member, said flexibility region formed by selectively removing a portion of the outer member to expose the inner member.

2. The device of claim 1, wherein the first material is selected from the group of materials consisting of stainless steel, gold, molybdenum, platinum, titanium, tungsten, Elgiloy, L605, MP35N, Ta-10W, 17-4PH, Aeromet 100, cobalt-chrome alloy, cobalt alloy, metal glass alloy, and refractory metal alloy.

3. The device of claim 1, wherein said second material comprises a shape-memory material.

4. The device of claim 1, wherein said second material comprises a superelastic material.

5. The device of claim 1, wherein said outer member further includes a polymeric coating.

6. The device of claim 1, wherein at least a portion of said outer member is electrically removed.

7. The device of claim 1, wherein at least a portion of said outer member is chemically removed.

8. The device of claim 1, wherein at least a portion of said outer member is mechanically removed.

9. The device of claim 1, wherein the composite medical device comprises an intravascular filter.

10. The device of claim 1, wherein the composite medical device comprises a stent or stent graft.

11. An intravascular filter device for placement within a body vessel, comprising:
a plurality of elongated legs each having a proximal end and a distal end, the elongated legs being secured together;
each of said plurality of elongated legs being formed of an outer member comprising a first material, and an inner member comprising a second material different from the first material.

12. The intravascular filter device of claim 11, wherein said plurality of elongated legs are rod members.

13. The intravascular filter device of claim 11, wherein said plurality of elongated legs are tubular members.

14. The intravascular filter device of claim 11, wherein said plurality of elongated legs are ribbon members.

15. The intravascular filter device of claim 11, wherein said plurality of elongated legs are configured to expand from a substantially straight position to an outswept position when placed within the body vessel.

16. The intravascular filter device of claim 11, wherein at least a portion of the distal end of said outer member is removed.

17. The intravascular filter device of claim 11, wherein said first material includes a radiopaque material.

18. The intravascular filter device of claim 11, wherein the first material is selected from the group of materials consisting of stainless steel, gold, molybdenum, platinum, titanium, tungsten, Elgiloy, L605, MP35N, Ta-10W, 17-4PH, Aeromet 100, cobalt-chrome alloy, cobalt alloy, metal glass alloy, and refractory metal alloy.

19. The intravascular filter device of claim 11, wherein the inner member is more elastic than the outer member.

20. The intravascular filter device of claim 11, wherein said outer member further includes a polymeric coating.

21. The intravascular filter device of claim 11, wherein said second material comprises a shape-memory material.

22. The intravascular filter device of claim 11, wherein said second material comprises a superelastic material.

23. The intravascular filter device of claim 11, wherein each of said plurality of elongated legs includes a hook region configured to engage the walls of the body vessel.

24. The intravascular filter device of claim 23, wherein said hook region is formed by removing at least a portion of said outer member.

25. The intravascular filter device of claim 23, wherein said hook region comprises a main section, a reversibly bent section, and a pointed tip section.

26. The intravascular filter device of claim 11, wherein at least a portion of said outer member is removed to form one or more zigzag regions along each of said plurality of elongated legs.

27. The intravascular filter device of claim 26, wherein said one or more zigzag regions are longitudinally offset from each other.

28. The intravascular filter device of claim 11, wherein at least a portion of said outer member is electrochemically removed.

29. The intravascular filter device of claim 11, wherein at least a portion of said outer member is chemically removed.

30. The intravascular filter device of claim 11, wherein at least a portion of said outer member is mechanically removed.

31. An intravascular filter device for placement within a body vessel, comprising:
an apical head; and
a plurality of elongated legs each having a proximal end and a distal end, the distal end of each of said plurality of elongated legs being secured to the apical head;
each of said plurality of elongated legs being formed of an elastic inner member and a stiff outer member, wherein a portion of said stiff outer member is removed to form a hook region along each elongated leg.

32. The intravascular filter device of claim 31, wherein said plurality of elongated legs are rod members.

33. The intravascular filter device of claim 31, wherein said plurality of elongated legs are tubular members.

34. The intravascular filter device of claim 31, wherein said plurality of elongated legs are ribbon members.

35. The intravascular filter device of claim 31, wherein said plurality of elongated legs are configured to bend or flex from a substantially straight position to an outswept position when placed within the body vessel.

36. The intravascular filter device of claim 31, wherein at least a portion of the distal end of said stiff outer member is removed.

37. The intravascular filter device of claim 31, wherein said stiff outer member includes a radiopaque material.

38. The intravascular filter device of claim 31, wherein the stiff outer member is formed of a material selected from the group of materials consisting of stainless steel, gold, molybdenum, platinum, titanium, tungsten, Elgiloy, L605, MP35N, Ta-10W, 17-4PH, Aeromet 100, cobalt-chrome alloy, metal glass alloy, and refractory metal alloy.

39. The intravascular filter device of claim 31, wherein said stiff outer member includes a polymeric material.

40. The intravascular filter device of claim 31, wherein said elastic inner member comprises a shape-memory material.

41. The intravascular filter device of claim 31, wherein said elastic inner member comprises a superelastic material.

42. The intravascular filter device of claim 31, wherein said hook region comprises a main section, a reversibly bent section, and a pointed tip section.

43. The intravascular filter device of claim 31, wherein at least a portion of said stiff outer member is removed to form one or more zigzag regions along each of said plurality of elongated legs.

44. The intravascular filter device of claim 43, wherein said one or more zigzag regions are longitudinally offset from each other.

45. The intravascular filter device of claim 31, wherein at least a portion of said stiff outer member is electrochemically removed.

46. The intravascular filter device of claim 31, wherein at least a portion of said stiff outer member is chemically removed.

47. The intravascular filter device of claim 31, wherein at least a portion of said stiff outer member is mechanically removed.

48. An intravascular filter device for placement within a body vessel, comprising:
an apical head; and
a plurality of elongated legs each having a proximal end and a distal end, the distal end of each of said plurality of elongated legs being secured to the apical head;
each of said plurality of elongated legs being formed of an elastic inner member and a stiff outer member, wherein a portion of said stiff outer member is removed to form a hook region and one or more zigzag regions along each elongated leg.

49. An intravascular filter device for placement within a body vessel, comprising:
an apical head having a proximal portion and a distal portion;
a plurality of elongated legs each having a proximal end and a distal end, the distal end of each of said plurality of elongated legs having a reduced diameter portion secured to the proximal portion of the apical head;
each of said plurality of elongated legs being formed of an elastic inner member and a stiff outer member, wherein a portion of said stiff outer member is removed to form a hook region and one or more zigzag regions along the length of each elongated leg, the one or more zigzag regions being longitudinally offset from each other.

50. A composite stent, comprising:
a plurality of threads formed from an outer member comprising a first material, and an inner member comprising a second material different from the first material, wherein the second material is more elastic than the first material; and
at least one flexibility region formed on said composite elongated member, said flexibility region formed by selectively removing a portion of the outer member to expose the inner member.

51. The composite stent of claim 50, wherein said plurality of threads are wire members.

52. The composite stent of claim 50, wherein said plurality of threads are tubular members.

53. The composite stent of claim 50, wherein said plurality of threads are ribbon members.

54. The composite stent of claim 50, wherein the stent is configured to self-expand when deployed in a body vessel.

55. The composite stent of claim 50, wherein said composite stent includes a middle portion and two end portions.

56. The composite stent of claim 55, wherein said at least one flexibility region is located at either of said two end portions.

57. The composite stent of claim 50, wherein said first material includes a radiopaque material.

58. The composite stent of claim 50, wherein the first material is selected from the group of materials consisting of stainless steel, gold, molybdenum, platinum, titanium, tungsten, Elgiloy, L605, MP35N, Ta-10W, 17-4PH, Aeromet 100, cobalt-chrome alloy, cobalt alloy, metal glass alloy, and refractory metal alloy.

59. The composite stent of claim 50, wherein said outer member further includes a polymeric coating.

60. The composite stent of claim 50, wherein said second material comprises a shape-memory material.

61. The composite stent of claim 50, wherein said second material comprises a superelastic material.

62. The composite stent of claim 50, wherein at least a portion of said outer member is electrically removed.

63. The composite stent of claim 50, wherein at least a portion of said outer member is chemically removed.

64. The composite stent of claim 50, wherein at least a portion of said outer member is mechanically removed.
